(19) United States
(12) Patent Application Publication
(10) Pub. No.: US 2016/0177285 A1
(43) Pub. Date: Jun. 23, 2016

(54) NOVEL MEANS TO DECREASE THE NEGATIVE EFFECTS OF SMOKING

(71) Applicant: Gerard VOERMAN, Brasschaat (BE)

(72) Inventors: Gerard VOERMAN, Brasschaat (BE); Friso Martijn VOERMAN, Brasschaat (BE)

(21) Appl. No.: 14/910,083

(22) PCT Filed: Aug. 5, 2014
(86) PCT No.: PCT/EP2014/066844

§ 371 Date: Feb. 4, 2016

(30) Foreign Application Priority Data
Aug. 5, 2013 (EP) PCT/EP2013/066415

(51) Int. Cl. C12N 9/48; A24B 13/00; A24F 47/00; C07K 14/81; A61K 9/00

(52) U.S. Cl. CPC C12N 9/48; C07K 14/811; A61K 9/007; A24F 47/002; A24B 13/00; A61K 38/00

(57) ABSTRACT

The present invention relates to the use of an elastase inhibitor, preferably fahsin for the treatment or prevention of emphysema, COPD or lung cancer. The elastase inhibitor is preferably administered through inhalation, preferably thorough inhalation of tobacco smoke. The invention also comprises smoking articles comprising such an elastase inhibitor.

### FIELD OF THE INVENTION

The invention relates to the field of health and medicine, more particularly in the field of preventing or trating lung diseases, such as COPD, emphysema and lung cancer, and alternatively in the field of overcoming problems by smoking, more specifically cigarettes, tobacco and other smoking materials for reducing the negative effects of smoke inhalation.

### BACKGROUND OF THE INVENTION

Smoking is bad for your health. Ever since the 1970's more and more evidence has been gathered about the negative influence of smoking, particularly smoking cigarettes, but also other forms of smoking, on the health of the smoker and the non-smoking bystanders. It is widely known, and has been established by numerous clinical experiments that cigarette smoke is playing a pivotal role in the deterioration of the elastine content in lung structures, such as trachea, bronchi, broncheoli, and alveolar systems. More specifically, emphesema is a local defect or rupture of alveolar walls.

Capillaries in the alveolar walls are the most important anatomic feature. They form an intertwining network and are supported by a delicate fibrous stroma enriched by elastic and reticulin fibres. Macrophages occur within the alveolar spaces, and form a defence mechanism of first order against invading bacteria. According to the “elastase:antielastase” hypothesis, cigarette smoke causes inflammation and subsequent release of proteolytic enzymes into the lung in excess of their natural inhibitors. In the absence of normal repair, proteolysis leads to tissue destruction and airspace enlargement. This elastase:antielastase hypothesis has dominated COPD research for nearly four decades. In 1963, Laurell and Eriksson (Laurell C B, Eriksson S. Scand J Clin Invest 1963, 15:132-140) reported an observation that patients who produced no or insufficient amounts of α-1-antitrypsin (α₁ AT), the major inhibitor of neutrophil elastase (NE), developed early onset emphysema. Soon thereafter, Gross et al (Gross P et al. Arch Environ Health 1965, 11:50-58) instilled papain, an elastase, into rat lungs, resulting in emphysema. Subsequently, investigators were able to induce emphysema by instilling other elastolytic enzymes, including human neutrophil elastase, into the airways of experimental animals (Senior R M et al. J Clin Invest 1980, 66:859-862; Janoff A et al. Am Rev Respir Dis 1977, 115:461-478; Snider G L et al. Am Rev Respir Dis 1984, 129:155-160). Together, these observations firmly established NE as the proteinase most likely responsible for tissue destruction in emphysema. The relation with the smoking of cigarettes has been firmly established by Shapiro et al. (Shapiro S et al. Am J Pathol. 2003 163(6):2329-2335).

The occurrence of lung cancer has undoubtedly been related to smoking as has been shown in numerous studies. There is no single form of lung cancer, and it may consist of bronchiogenic carcinoma, alveolar carcinoma, bronchial adenoma, and mesenchymal tumors. Especially bronchiogenic carcinoma has been related to (cigarette) smoking. The anatomical changes seen with this cancer are changes to the epithelium, such as loss of hair cells, basal cell hyperplasia, squamous cell metaplasia and atypical cell structures. Although elastase inhibition has not been shown to be influenced the carcinogenic activity of the chemical compounds, like polycyclic aromatic hydrocarbons, that are present in cigarette smoke and are proven to be carcinogenic, it is hypothesized that elastase inhibition can influence the cascade of events that accompany carcinogenesis, such as the effects of the immune component of cancer.

Although elastase inhibitors have been suggested for therapy of emphysema (e.g. in Koraki, T. et al., 2002, Am. J. Resp. Crit. Care med. 166:496-500; Wright, J. et al., 2003, Eur. Resp. J. 22:77-81), up till now no commercial use of these compounds in the field of lung emphysema has been achieved.

Also there is need for new proteinase inhibitors that have specificity for particular proteinases, such as elastase, trypsin, chymotrypsin, cathepsin G, and the like.

### SUMMARY OF THE INVENTION

The current inventor now has found that fahsin may be used in the prevention of lung disease, selected from the group of emphysema, COPD and lung cancer. Such a use in therapy or prophylaxis of lung disease, particularly emphysema, can preferably be effected through inhalation, specifically through inhalation of smoke, more particularly tobacco (e.g. cigarette) smoke.

The invention also comprises a smoking article, such as a cigarette, including an e-cigarette, pipe tobacco, cigar or joint, for use in the therapy or prophylaxis of emphysema. Preferably said smoking article comprises an elastase inhibitor, preferably selected from the group of fahsin, guamerin, piguamerin, hirustasin, bdellastasin and mutants of guamerin, piguamerin, hirustasin and bdellastasin that contain a leucine residue after the 6^{th} cysteine residue. Further preferably said elastase inhibitor is included in tobacco or cannabis, either as a blend or as a protein expressed by said tobacco or cannabis. Alternatively, the cigarette is a filter cigarette or an e-cigarette and the elastase inhibitor is present in the filter. Further alternatively, the smoking article is a cigarette or a joint and the elastase inhibitor is present in the cigarette paper.

In a further embodiment, the elastase inhibitor is recombinantly produced fahsin.

Also comprised in the invention is transgenic tobacco or cannabis comprising an elastase inhibitor, preferably fahsin. Consequently, the invention comprises a cigarette comprising such transgenic tobacco or cannabis.

Further part of the invention is a method to prevent or reduce emphysema, COPD or lung cancer comprising smoking an smoking article according to the invention.

Also an embodiment of the present invention is a method to prevent or reduce emphysema, COPD or lung cancer comprising inhaling an elastase inhibitor, preferably fahsin, more preferably recombinant fahsin.

The invention also comprises a method to improve the lung function of smokers by administration of an elastase inhibitor, preferably by administration of said inhibitor by inhalation. Preferably in such a method said inhalation is inhalation of smoke wherein said smoke comprises said inhibitor.

In a further embodiment, the invention provides new mutants of fahsin, guamerin, piguamerin, hirustasin and bdellastasin.

### LEGENDS TO THE FIGURES

FIG. 1: Alignment of the primary amino acid sequence of five different antistasin-type serine proteinase inhibitors. The similarly spaced cysteine residues in the proteins are indicated in bold. The reactive site (P1) amino acid residue, reflecting the specificity of the inhibitor, is underlined.

FIG. 2. Inhibition of human neutrophil elastase by different mutants of guamerin. Indicated on the X-axis is the residue of the mutant at the P1 position (met is the wild-type), Blanco is only substrate and max is substrate +elastase. The bars represent the time after start of incubation. The y-axis gives the A405-A540 difference measured.

FIG. 3. Inhibition of cathepsin G inhibition by mutants of fahsin in different concentrations. Indications of mutants and Y-axis similar as in FIG. 2.

FIG. 4. Inhibition of various proteinases by mutants of guamerin. A: cathepsin G, B: chymotrypsin, C: elastase, D: trypsin, E: plasmin, F: thrombin

### DETAILED DESCRIPTION

One of the recently found elastase inhibitors is fahsin, which is derived from the Nile leech Limnatis nilotica (De Bruin, E. et al., FEMS Yeast Res. 5:1069-1077, 2005; WO 96/13585). It has been demonstrated in this publication that fahsin is a proteinase that is specific for human neutrophil elastase (hNE) and leaves other important blood-derived serine proteases, such as plasmin, thrombin, tPA, coagulation factors VIIa, Xa, XIa and XIIa untouched. This makes it an ideal candidate for the present invention.

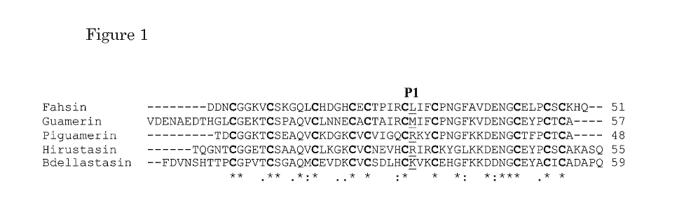

Further, although fahsin is a peptide compound, it is easy to produce with the aid of recombinant techniques and it has proven to be very stable. The amino acid sequence (GenBank DQ097891.1) and the nucleotide sequence coding for said amino acid sequence (GenBank AAY85799.1) has been provided in FIG. 1. As is shown in De Bruin et al., (supra) fahsin is similar to other antistasin-type proteinase inhibitors by having a consensus sequence with 10 cysteine residues at specific distances:

C (X₄) CS (X₄) C (X₄) CXC (X₄) C L (X₃) C (X₆) DXNGC (X₃) CXC

in which X may be any amino acid, and C, L, N, G and C have their normal meaning in the nomenclature of amino acids.

The specificity for NE is attributed to the leucine residue behind the 6^{th} cysteine residue. It is submitted that peptides having the above consensus sequence may be used in the present invention and will have an NE-inhibitory effect. Next to fahsin also other antistasin type serine proteinase (NE) inhibitors maybe used in the present invention. Examples are guamerin, piguamerin, hirustasin and bdellastasin. The amino acid sequences for these compounds are:
guamerin (Hirudo nipponia): vdenaedthg lcgektcspa qvclnnecac taircmifcp ngfkvdengc eypctca
Piguamerin (Hirudo nipponia): tdcggktcse aqvckdgkcv cvigqcrkyc pngfkkdeng ctfpctca
Hirustasin (Hirudo medicinalis): tqgntcgget csaaqvclkg kcvcnevhcr irckyglkkd engceypcsc akasq
Bdellastasin (Hirudo medicinalis: fdvnshttpc gpvtcsgaqm cevdkcvcsd lhckvkcehg fkkddngcey acicadapq

Of these alternatives to fahsin especially preferred is a mutated guamerin, wherein the methionine residue after the 6th cysteine residue is changed into a leucine residu: vdenaedthg lcgektcspa qvclnnecac tairclifcp ngfkvdengc eypctca. It has been shown (results not shown) that such a mutated guamerin is insensitive to both chemical and biological oxidation and further this mutated protein also appeared to be a strong inhibitor of NE like the wild-type fahsin molecule. It is believed that changing this specific residue (see FIG. 1), which in the other molecules mentioned above is an arginine residue (piguamerin and hirustasin) or a lysine residue (bdellastasin) into a leucine residue also provides mutant proteins that have an improved reactivity towards NE and also are more stable than the wild-type proteins.

As such, the mutant proteins having a leucine residue after the 6th cysteine residue also form part of the invention.

Other mutants that have been made while studying the mutants that are applicable in the present invention comprises a number of fahsin mutants, in which the P1 site (i.e. the residue following the 6th cysteine residue) has been changed. Several mutants were made with P1=Arg, P1=Ile, P1=Met and P1 is Val. These mutants were made via site-directed mutagenesis. It appeared that Fahsin-Ile (i.e. the residue following the 6th cysteine residue is Ile) is a very specific inhibitor of elastase and does not inhibit chymotrypsin, cathepsin G and proteinase 3. This means that this mutant is very suitable for diseases in which specifically elastase is a causing factor, such as emphysema and psoriasis. Also, this mutant could be very well suited for arthritis, gingivitis, periodontitis and other inflammatory conditions that are associated with tissue destruction caused by the enzyme human neutrophil elastase (HNE).hence, the invention also covers use of this Fahsin-Ile mutant as a therapeutic compound, especially for the treatment of inflammatory diseases that are related to neutrophil elastase, and in particular for emphysema, periodontitis, arthritis and the like. It is submitted that for the treatment of emphysema and periodontitis the administration preferably is given orally. For emphysema treatment administration may be given by any form of inhaler, but advantageously through an e-cigarette as described herein. For periodontitis also an e-cigarette delivery may be used, but the compound may also be provided in toothpaste, chewing-gum or other administration forms that provide for release of the compound in the oral cavity.

Also the Fahsin-Val and the Fahsin-Met mutant may be used as elastase-inhibitors in the same way as indicated above for Fhasin-Ile, although their effect is less specific than the Fahsin-Ile mutant and the wild-type fahsin.

A second very useful fahsin mutant is Fahsin-Arg. This compound, although it only differs in one amino acid from wild-type fahsin does not specifically inhibit elastase, but surprisingly it is an excellent inhibitor of trypsin (and it also inhibits the coagulation factors Xa, XIa and XIIa). Because of these effects, Fahsin-Arg is deemed suitable for inhibition of coagulation and fibrinolysis. Also, Fahsin-Arg may be used in the therapy of pancreatitis. Fahsin-Arg is also a stronger cathepsin G inhibitor than the other fahsin mutants. This means that it can also be used as a cathepsin g inhibitor, and thus that it would be useful to treat or prevent inflammation, especially where inflammation leads to edema, to treat or prevent photoaging. Also, it enhances the antithrombotic effects of Fahsin-Arg.

For guamerin, next to the above discussed Leu mutant, also other mutants have been made with Ile, Arg, Lys or Val at the P1 position (see FIG. 1). As discussed above, the Leu mutants was the most effective in inhibition of human neutrophil elastase, while also the wild-type (with Met at the P1 position) showed some effects. The other three mutants were less effective. However, the Arg mutant appeared to be the best inhibitor of chymotrypsin, with the Leu mutant coming second. These two were also the best inhibitors of cathepsin G. The Lys mutant, however, proved to be a specific inhibitor of trypsin and plasmin, where for the other mutants only guamerin-Arg could show some effects. Thrombin was hardly inhibited by any of the mutants.

Further, the previously contemplated application of elastase inhibitors in the field of lung diseases such as COPD and emphysema, has been focused on the application after emphysema was already established. It will be clear that the application of an elastase inhibitor in cigarettes is meant to prevent the onset of emphysema or to inhibit further progress of the emphysema. Thus, also the present invention provides for the prevention of emphysema or the prevention of the progress of emphysema by fahsin.

Such an application is especially useful to prevent or to decrease the harmful effects of smoking, especially of smoking cigarettes. Moreover, it has been demonstrated (Kozumi, F. et al., 1999, Clin. Pharmacol. Ther, 66:501-508) that uptake of an elastase inhibitor in lungs of smokers may be increased with respect to the uptake by non-smokers, which aids in making such a therapy very suitable to combat the harmful effects of smoking.

An NE inhibitor to the present invention can be used in an inhaler to prevent or treating diseases, such as COPD, emphysema and lung cancer. Further, administration of an NE inhibitor can be used to ameliorate or bring relief in conditions where the lungs are clotted or long-function is impaired because of other means. Such conditions include asthma, pneumonia caused by bacteria or other micro-organisms, such as Pneumococcus sp., Staphylococcus sp., Haemophilus influenza, Pseudomonas aeruginosa, Moraxella catharalis, Mycoplasma sp., Chlamydophilia pneumonia, legionella pneumophila, respiratory Syncitial Virus (RSV), adenovirus, Chlamydia spp, Aspergillus sp., common cold, destruction or impairment of lung tissue by asbestosis, air pollution, and the like. Ideally, inhalation of an NE inhibitor, such as fahsin can be delivered through inhalation, in particular by inhalation through an inhaler or inhalation through an electronic cigarette (e-cigarette).

For smokers, an NE inhibitor according to the present invention, especially fahsin, and particularly recombinant fahsin, can be included in smoking articles in any conceivable way. First of all, it is possible that the peptide is expressed recombinantly in the tobacco or other plant material (cannabis) that is contained in the cigarette. For such recombinant production a similar expression construct as has been used in the examples for obtaining recombinant expression in Pichia pastoris may be used, but of course then adapted to expression in plants.

There are multiple ways in which a recombinant nucleic acid can be transferred to a plant cell, for example Agrobacterium mediated transformation. However, besides by Agrobacterium infection, there are other means to effectively deliver of DNA to recipient plant cells when one wishes to practice the invention. Suitable methods for delivering DNA to plant cells are believed to include virtually any method by which DNA can be introduced into a cell, such as by direct delivery of DNA such as by PEG-mediated transformation of protoplasts, by desiccation/inhibition-mediated DNA uptake (Potrykus et al., Mol. Gen. Genet., 199:183-188, 1985), by electroporation (U.S. Pat. No. 5,384,253), by agitation with silicon carbide fibers (Kaeppler et al., 1990; U.S. Pat. No. 5,302,523; and U.S. Pat. No. 5,464,765), and by acceleration of DNA coated particles (U.S. Pat. No. 5,550,318; U.S. Pat. No. 5,538,877; and U.S. Pat. No. 5,538,880). Through the application of techniques such as these, cells from virtually any plant species may be stably transformed, and these cells developed into transgenic plants.

In case Agrobacterium mediated transfer is used, it is preferred to use a substantially virulent Agrobacterium host cell such as A. tumefaciens, as exemplified by strain A281 or a strain derived thereof or another virulent strain available in the art. These Agrobacterium strains carry a DNA region originating from the virulence region of the Ti plasmid pTiBo542 containing the virB, virC and virG genes. The virulence (vir) gene products of A. tumefaciens coordinate the processing of the T-DNA and its transfer into plant cells. Vir gene expression is controlled by virA and virG, whereby virA upon perception of an inducing signal activates virG by phosphorylation. VirG, in turn, induces the expression of virB,C,D,E. These genes code for proteins involved in the transfer of DNA. The enhanced virulence of pTiBo542 is thought to be caused by a hypervirulent virG gene on this Ti plasmid (Chen et al. Mol. Gen. Genet 230: 302-309, 1991).

After transfer of a nucleic acid into a plant or plant cell, it must be determined which plants or plant cells have been provided with said nucleic acid. This is for example accomplished by using a selectable marker or a reporter gene. Among the selective markers or selection genes that are most widely used in plant transformation are the bacterial neomycin phosphotransferase genes (nptI, nptII and nptIII genes) conferring resistance to the selective agent kanamycin, suggested in EP131623 and the bacterial aphIV gene suggested in EP186425 conferring resistance to hygromycin. EP 275957 discloses the use of an acetyl transferase gene from Streptomyces viridochromogenes that confers resistance to the herbicide phosphinotricin. Plant genes conferring relative resistance to the herbicide glyphosate are suggested in EP218571. The resistance is based on the expression of a gene encoding 5-enolshikimate-3-phosphate synthase (EPSPS) that is relatively tolerant to N-phosphomethylglycine. Certain amino acids such as lysine, threonine, or the lysine derivative amino ethyl cysteine (AEC) and tryptophan analogs like 5-methyl tryptophan can also be used as selective agents due to their ability to inhibit cell growth when applied at high concentration. In this selection system expression of the selectable marker gene results in overproduction of amino acids by transgenic cells which permits the transgenic to grow under selection. Suitable examples of reporter genes are beta-glucuronidase (GUS), beta-galactosidase, luciferase and green fluorescent protein (GFP).

Alternatively, transformants can be detected by assaying for the presence of the nucleic acid encoding fahsin or the fahsin protein expressed said nucleotide sequence.

As an alternative to Agrobacterium transformation, Kościańska and Wypijewski (2001, Acta Biochim. Polon. 48-3:657-661) presented electroporation approaches for intact BY-2 tobacco cultured cells as exemplified by expression of a plasmid expressing the reporter Green Fluorescent Protein (GFP). The electroporation procedure consisted of inducing plasmolysis of the cells for 15-20 minutes in a buffer system containing 5 mM CaCl₂, 10 mM NaCl, 8.7% glycerol, 0.4M sucrose and 10 mM pipes buffer at pH 6.8 in the presence of 30 μg of the plasmid. Then the cells were subjected to vacuum after which they were incubated on ice before they were subjected to electroporation by applying a pulse at 2 kV/cm which lasted 80 μs. After the electro pulse the cells were again incubated on ice for 10 minutes, at room temperature for 10 minutes after which the cells were deplasmolysed by adding BY medium without sucrose to reduce the sucrose concentration from 0.4M to 0.05M in three steps. The deplasmolysed cells were then transferred to BY medium and GFP expression was monitored in subsequent days. The transfection efficiency was determined to be 50%, while the vitality of the cells was 70% after electroporation but decreasing in following days. Thus it seems that in this procedure the treatment to introduce the DNA has a negative effect on vitality and regeneration capacity of the cells.

A more elegant method was proposed by Chen, C.-P. et al. (2007, FEBS Lett. 581:1891-1897) using a poly-arginine based peptide for the delivery of both the GFP protein and GFP expression vector in intact roots of mung bean and soybean roots. In this example a nona-Arg peptide was produced as the carrier and 10 μg was pre-incubated with 10 μg of plasmid in a total volume of 50 μl PBS for 30 minutes at 37° C. Subsequently roots of mung bean and soybean were immersed for 30 minutes in the DNA-peptide solution, and washed afterwards. Expression of GFP was monitored and showed to occur between 24 hours and 48 hours after treatment throughout the entire root.

Further alternative methods to express the nucleic acid encoding an elastase inhibitor according to the invention may be used.

When fahsin is recombinantly produced in tobacco or cannabis, this tobacco or cannabis can be used to be included in cigarettes, either by blending it with other tobacco or by using it as such. It may also be added to tobacco or other smoking material by soaking the tobacco in a solution of the elastase inhibitor and then drying the tobacco.

Fahsin, or any other elastase inhibitor according to the present invention, may also be included in the cigarette paper that is used for rolling the cigarettes. For this purpose, the cigarette paper may be produced from pulp from recombinant plants that are able to express fahsin, e.g. transgenic rice, or fahsin may be added to the pulp during the process of preparing the cigarette paper. Alternatively, fahsin may be coated onto the cigarette paper after production and before rolling the cigarette.

Further, an elastase inhibitor may be added to the smoking material during preparation of said material. For this it may be blended with the tobacco, either as a protein powder or encapsulated in a carrier material. Also, it may be added to the filter material in filter cigarettes. In the experimental section it has been shown that insertion of rFahsin containing acrylate beads into the filters of cigarettes produced a significant amelioration of the lung function of the test subjects. Also, the elastase inhibitor may be included into the filter material by soaking said material in a solution of the inhibitor end drying it before the production of cigarette filters.

Lastly, an elastase inhibitor may be inhaled concomitantly with (cigarette)smoke if the smoke is inhaled through a material or a pipe in which said inhibitor is released. This can be an additional filter material, but it can also be a carrier material that slowly releases the elastase inhibitor that by the user e.g. is applied in a cigarette pipe before starting smoking.

Of course the smoking article in which the elastase inhibitor is included may be any smoking article, such as a cigarette, a cigar, a cigarillo, a pipe, a joint, a waterpipe or any other smoking material. Preferably, the smoking article is a cigarette, since that is mostly used and since that has been considered as the most relevant in the cause of lung diseases.

The elastase inhibitor that is used in a smoking material is preferably fahsin, more preferably recombinant fahsin. Fahsin has the major advantage that it is extremely heat stable and thus will not be deteriorated by the hot smoke. In one test fahsin has been hated to 123° C. without appearance of a melting curve. After this high temperature treatment the protein did not loose in activity. The elastase inhibitor may be present in said smoking article in a concentration of 0.001 to 100 mg/kg smoking material, but preferably in a concentration of 0.001 to 50 mg/kg smoking material. From our experiments it has appeared that the minimal inhibitory concentration (MIC) is 4 μg/1 million PMN's per 15 minutes. However, since the MIC depends largely on disease gravity it is possible to use smoking material having different amounts of elastase inhibitor. In this way, several grades of smoking material (light-medium-strong) can be provided, all having the appropriate amount of elastase inhibitor that would suffice for treating or preventing the effects of tobacco smoke.

The elastase inhibitor may also be applied to the lungs without inhalation of smoke, e.g. by use of a standard inhaler or vaporizer that is normally used for administration of pharmaceutical compounds to the bronchi, bronchiole or alveoli. To this extent, the elastase inhibitor, preferably fahsin or guamerin-Leu, may be present in said inhaler in any acceptable pharmaceutical formulation, such as a dry powder, or in a solution. Especially an inhaler that comprises a vaporizer in which a solution or suspension that contains the elastase inhibitor is a solution which may form an aerosol, is preferred. An important parameter for an efficient aerosol delivery producing a systemic therapeutic effect is the particle size distribution in the aerosol cloud. When the formulation is in the form of suspension, the particle size of the cloud is dominated by the particle size of the suspended drug. When the formulation is in the form of solution, the volumetric contribution of suspended drug particles is absent and much finer liquid droplets clouds, largely defined by the drug concentration in the solution, are generated. When the medicament is delivered to the lungs through an aerosol inhaler so as to be induced into the capillaries, the particles should be small enough to be delivered to the lungs and to be absorbed into the bloodstream upon inhalation, i.e. of a size advantageously comprised between about 0.5 μm and 2.5 μm. Particles smaller than 0.5 μm are not therapeutically useful as they are exhaled again. It is submitted that the skilled person will be able to produce an effective pharmaceutical formulation with the elastase inhibitor of the invention for use in an inhalation or vaporiser device.

Next to these more or less medicinal inhalers and vaporisers, the elastase inhibitor of the invention may also be included in vaporizers that are used for moistening the air or bringing scents into the air. In such a case the concentration of the elastase inhibitor may be low. Continuously refreshing the air in a house then will allow for a constant presence of a small amount of elastase inhibitor in the air and thus for a constant inhalation dose for the inhabitants of the house. Of course, such a use is not confined to a house, but it can also be applied in a car, in shops, in offices, in public buildings and the like.

One specific form of an inhaler is an electronic cigarette or e-cigarette. An e-cigarette or personal vaporizer (PV) is an electrical charge powered vaporizer which simulates tobacco smoking by producing an aerosol that resembles smoke. It generally uses a heating element known as an atomizer, that vaporizes a liquid solution known as e-liquid. E-liquids usually contain a mixture of propylene glycol, vegetable glycerin, nicotine, and flavorings while others release a flavored vapor without nicotine. For the present invention an e-cigarette is very advantageously used because the elastase inhibitor may be solved in the E-liquid and thus contained in the aerosol that is produced for inhaling. The solution is often sold in bottles or pre-filled disposable cartridges, or as a kit for consumers to make their own E-liquids. Components are also available individually and consumers may choose to modify or boost their flavor, nicotine strength, or concentration with various offerings.

In a personal vaporizer, the atomizer system may be represented in the form of a so-called ‘cartomizer’, which consists of an atomizer surrounded by a liquid-soaked poly-foam that acts as an e-liquid holder. Cartomizers can be used on their own or in conjunction with a tank that allows more e-liquid capacity. When used in a tank, the cartomizer is inserted in a plastic, glass or metal tube and holes or slots have to be punched on the sides of the cartomizer to allow liquid to reach the coil. Clearomizers or “clearos”, not unlike cartotanks, use a clear tank in which an atomizer is inserted. Unlike cartotanks, however, no poly-foam material can be found in them. There are a lot of different wicking systems employed inside of clearomizers to ensure good moistening of the wick without flooding the coil. Some rely on gravity to bring the e-liquid to the wick and coil assembly (bottom coil clearomizers for example) whereas others rely on capillary action and to some degree the user agitating the e-liquid while handling the clearomizer (top coil clearomizers). A rebuildable atomizer or an RBA is an atomizer that allows the user to assemble or “build” the wick and coil themselves instead of replacing them by an off-the-shelf atomizer “head”. They also allow the user to build atomizers at any desired electrical resistance. The materials needed to “rebuild” the atomizers are usually much cheaper than the usual prefabricated replaceable wick and coil assemblies destined to clearomizers. These rebuildable atomizers are divided into two main categories; rebuildable tank atomizers (RTA's) and rebuildable dripping atomizers (RDA's).

Rebuildable tank atomizers or RTA's are similar to clearomizers in that they use a tank or container to hold and bring liquid to the coil. They usually hold a lot more e-liquid than their RDA counterparts.

Rebuildable dripping atomizers or RDA's on the other hand lack the container section and hold very little liquid compared to RTA's but are usually a lot smaller. They usually consist only of an atomizer “building deck” which can accept one or more coils and a “top cap” to cover the coils where a mouth piece can be attached. The user needs to manually keep the atomizer wet by dripping liquid on the bare wick and coil assembly

### EXAMPLE 1

Production and Characterization of Recombinant Fahsin

Production and purification of fahsin was performed as described in De Bruin, E. et al., FEMS Yeast Res. 5:1069-1077, 2005. In short, a synthetic fahsin gene was constructed by overlap extension PCR of four long oligonucleotides, codon usage optimized for the host Pichia pastoris:

FA-1:
5′-GGGGTATCTCTCGAGAAAAGAGACGACAACTGTGGTGGT
AAGGTTTGTTCTAAGGGTCAA-3′
FA-2:
5′-AATCAAACATCTAATTGAGTACACTCACAGTGACCGGTC
GTGACACAATTGACCCTTAGAACAAAC-3′
FA-3:
5′-CCAATTAGATGTTTGATTTTCTGTCCAAACGGTTTCGCT
GTTGACGAGAACGGTTGTGAG-3′
FA-4:
5′-GCTGGCGGCCGCTCATTGGTGCTTACAAGAACATGGCAA
CTCACAACCGTTCTCGTC-3′

After cloning of the PCR product using the pGEMT-easy cloning kit (Promega, Madison, Wis., USA) and subsequent DNA sequencing, the proper gene was cloned into the Pichia vector pPIC9, using the XhoI and NotI restriction endonucleases (Invitrogen, Carlsbad, Calif., USA).

P. pastoris GS115 (his4, see Cregg, J. et al., Mol. Cell. Biol. 5:3376-3385, 1985) was transformed by electroporation. Prior to transformation, plasmid pPIC9Fahsin was linearized with Sall (Invitrogen). After growth for 3 days on selective plates at 30° C., several colonies were selected for PCR confirmation using the vector primers 5ÁOX1 and 3ÁOX1 (Invitrogen).

After selection of rFahsin producing P. pastoris transformants, fermentations were conducted in a 5 liter BioFlo 3000 fermentor (New Brunswick Scientific, Edison, N.J., USA) in minimal basal-salt medium supplemented with 0.2% (v/v) PTMA₁-trace salts (Invitrogen). Methanol fed-batch fermentations (Potter, K. et al., Protein Expr. Purif. 19:393-402, 2000) were performed and rFahsin was purified from the fermentation broth using overnight dialysis against 20 mM Tris-buffer, pH 8,0. The rFahsin was separated using anion-exchange chromatography on a SP Sepharose FF column and eluted using a 1 M NaCl in citrate buffer (20 mM, pH 4.0) on Äkta explorer (GE Healthcare). With a chromogenic assay the activity of rFahsin containing chromatography fractions on NE was determined and active fractions were pooled and subsequently dialysed against 20 mM Tris-HCl, pH 8.0 to remove the NaCl. In a last anion exchange chromatography step on Q-Sepharose Fast Flow or Q-Sepharose High Performance substantially pure (>90%) as determined by HPLC (C8 reverse phase) was obtained.

Several characteristics of rFahsin were measured:
Incubation of rFahsin with a solution of 0.32% (w/v) pepsine and 10 mM HCl (pH 2.0) to mimic conditions encountered during gastrointestinal passage showed that rFahsin was completely inactivated.
Incubation of rFahsin with 10 mM HCl (pH 2.0) only did not affect the activity. Accordingly, inactivation was caused by pepsine, which was even capable of completely inactivating rFahsin at a ten times lower dosage.
rFahsin is extremely heat stable. After heating to 123° C. for one hour rFahsin did not loose its NE inhibiting activity. This characteristic makes it extremely suitable for delivery by (cigarette) smoke.
pH stability. After incubation during 48 hours at 60° C. at a pH of 2.0, 4.0, 6.0, 8.0 or 10.0 no specific activity appeared to be lost.
Incubation of rFahsin with equimolar concentrations of DDT did diminish the specific activity of rFahsin.
rFahsin inhibits NE by forming rFahsin-NE complexes, just like the natural human antagonist of NE, α1-antitrypsin (AAT). However, in contrast to AAT-NE complexes the rFahsin-NE complexes do not show any pro-inflammatory activity and probably are not quickly cleared from the body.
rFahsin is, in contrast to AAT, resistant against chemical and biological oxidation. This is an important advantage since during chronic inflammations, like those that occur with COPD, many activated neutrophils are present that cause the formation of reactive oxygen species (ROS). Further, rFahsin is not affected by chemical oxidation due to oxygen or active compounds in smoke.
rFahsin is capable of neutralizing human NE that is released due to stimulation of neutrophils with f-MLP (N-formyl-methionyl-leucyl-phenylalanine). rFahsin is also capable of neutralizing elastase activity ex vivo, i.e. in the gingival fluid of patients. Also, rFahsin appeared to be stable up to 72 hours in this gingival fluid.

### EXAMPLE 2

In Vitro Cytotoxicity Test with Purified Fahsin

L-929 mouse fibroblast cells (BioWhittaker, #3C0840) were stored as frozen stock cultures in liquid nitrogen. For the experiments they were grown in Dulbecco's modified Eagle medium (DMEM) supplemented with heat-inactivated calf serum (10% v/v), non-essential amino acids (1% v/v), L-glutamine (2 mM) and gentamicin (50 μg/ml). The cells were routinely cultured in a humidified incubator at 37° C.

Near-confluent L-929 cell cultures were harvested by trypsinization and resuspended in culture medium. The number of cells was counted using a Biirker-Türk counting chamber.

Four test samples of recombinantly produced protein (two forms of rFahsin, both purified in two different ways) were freeze-dried and before use solved in 500 μl culture medium, resulting in a concentration of about 1.59-3.64 mg/ml, sterilized and serial diluted.

Determination of cytotoxicity was performed by using the MTT assay (Mosmann, T., J. Immunol. Meth. 65:55-63, 1983). This assay determines the viability of cells by assessing their metabolic capability to reduce MTT to its corresponding MTT-formazan product. Briefly, the cells were incubated for 1 hour with 100 μl culture medium containing 0.5 mg MTT/ml. After incubation MTT medium is carefully removed and the MTT-formazan product is extracted for at least 1 hour using 1 ml DMSO. Absorbance is measured at a wavelength of 540 nm and a reference wavelength of 655 nm using a Biorad multi-well plate reader. As negative control culture medium without rFahsin was used, while as positive control a solution of 0.1% SDS was applied.

Results of the four test samples and the control did show that none of the recombinant fahsin samples induced cytotoxic effects (only highest concentrations of fahsin shown, table 1). Also no morphological changes of the L-929 cells were observed.

TABLE 1
Cytotoxic effects of recombinant fahsin

| | Concentration | Relative MTT |
|---|---|---|
| Sample | [μg/ml] | conversion (%) |
| rFahsin-1a | 3640 | 143.2 |
| rFahsin-1b | 2580 | 129.5 |
| rFahsin-2a | 2230 | 104.3 |
| rFahsin-2b | 1590 | 171.8 |
| medium | 0 | 100.0 |
| SDS | 0.1% | 0.3 |

### EXAMPLE 3

Effect of Smoking of Fahsin on Lung Function

14 persons, 8 men of 44-71 years old and 6 women of 44-69 years old) were followed while smoking cigarettes that contained fahsin. For this test fahsin containing cigarettes were prepared by solving recombinant fahsin in water and packaging this solution in acryl beads. The acryl beads were then manually inserted into the filter material of normal cigarettes by using miniscalpel and forceps. This resulted in a fahsin content per cigarette of about 0.04 μg/cigarette (ten times MIC, 1200 cigarettes/kg smoking material).

Smoking habit of the experimental subjects varied from about 10 to about 25 cigarettes a day. The lung function of the participants was tested with an FEV1 test (Donahue, J. F., COPD 2:111-124, 2005) at the onset of the experiment and at 4, 6 and 12 weeks after start of the experiment. This FEV1 was measured with an asthma monitor (Asma-1, Vitalograph, Buckingham, UK). All subjects had been smoking for several years before start of the experiment and their lung function already appeared to be less than considered normal for persons of comparable age and sex. As can be seen from Table 2, the FEV1 values generally improved over time when smoking fahsin containing cigarettes and a mean increase of 16% was found.

The experiment also shows that fahsin is still active after being heated by the cigarette smoke.

| | | | | | | Difference |
|---|---|---|---|---|---|---|
| | | | | | | after 12 |
| Sex | Age | Baseline | 4 weeks | 6 weeks | 12 weeks | weeks |
| M | 46 | 2.45 | 2.55 | 2.90 | 3.05 | +24% |
| F | 44 | 3.10 | 3.20 | 3.20 | 3.15 | +2% |
| F | 51 | 2.10 | 2.10 | 2.05 | 2.65 | +26% |
| F | 56 | 2.60 | 2.85 | 2.90 | 3.15 | +21% |
| M | 57 | 3.35 | 3.65 | 3.60 | 3.80 | +13% |
| M | 45 | 3.00 | 2.95 | 3.45 | 3.25 | +8% |
| M | 63 | 2.00 | 2.10 | 2.30 | 2.75 | +38% |
| M | 71 | 1.80 | 2.00 | 2.30 | 2.45 | +36% |
| M | 69 | 1.90 | 1.95 | 2.20 | 2.15 | +13% |
| F | 69 | 1.70 | 1.80 | 1.85 | 2.05 | +19% |
| F | 46 | 3.30 | 3.20 | 3.35 | 3.25 | −2% |
| M | 47 | 3.60 | 3.80 | 3.85 | 3.75 | +4% |
| F | 61 | 2.40 | 2.40 | 2.75 | 2.80 | +16% |
| M | 64 | 2.65 | 2.55 | 2.65 | 2.90 | +9% |

### EXAMPLE 4

Fahsin Mutants

Fahsin mutants with different amino acids at the P1 position (i.e. the Leu residue after the 6th cysteine residue) were made through site-directed mutagenesis in the strain that was used for producing the recombinant fahsin (Example 1, P. pastoris GS115). They were teste on several protein assays for testing the activity on other (serine) proteinases. As an example the effects of these mutants on cathepsin G is shown in FIG. 3.

### EXAMPLE 5

Guamerin Mutants

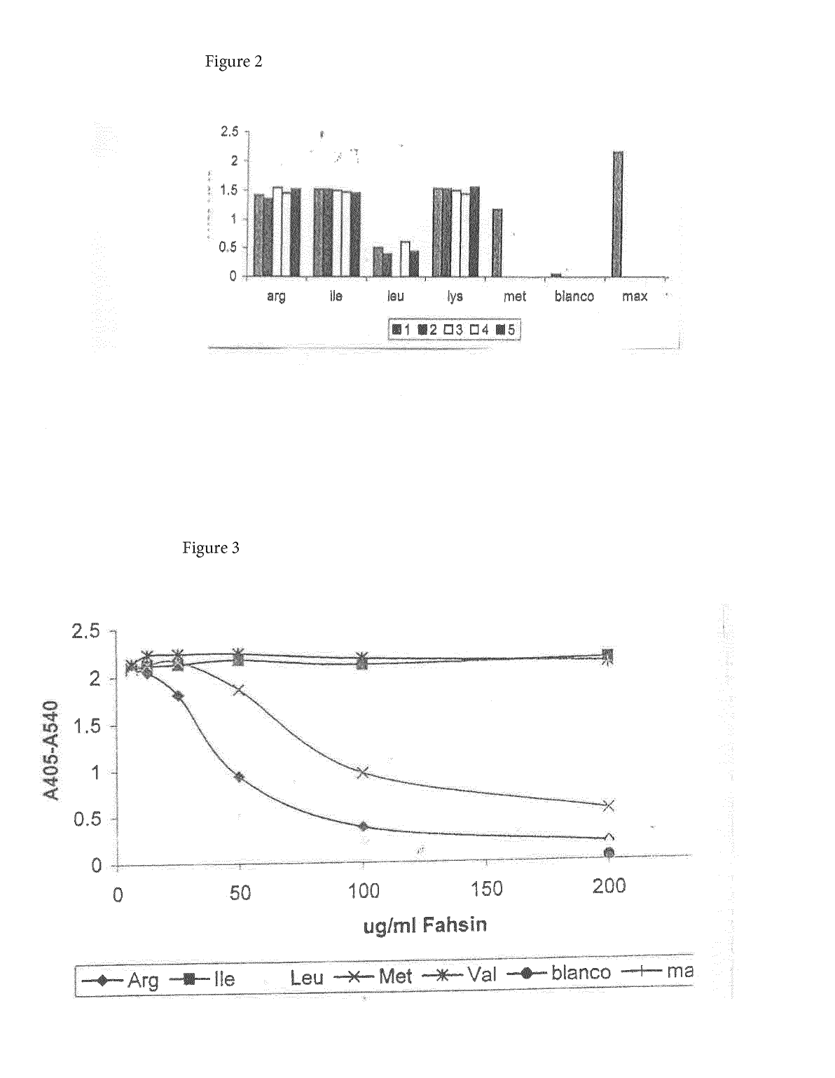

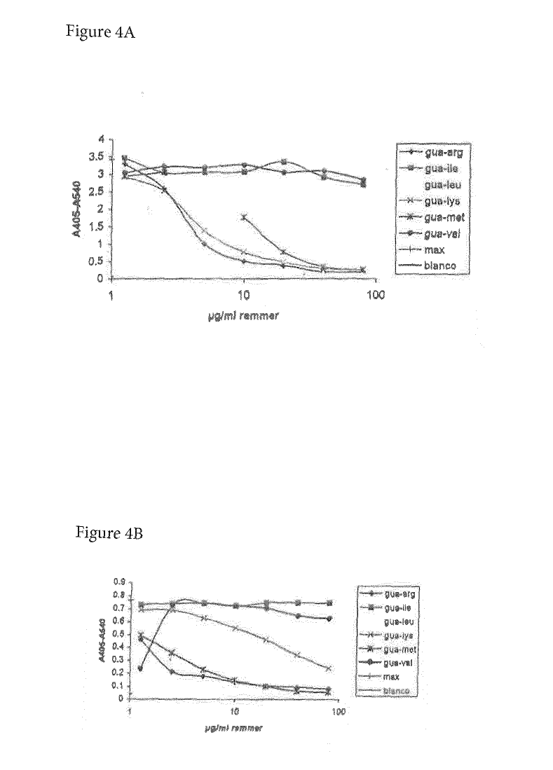

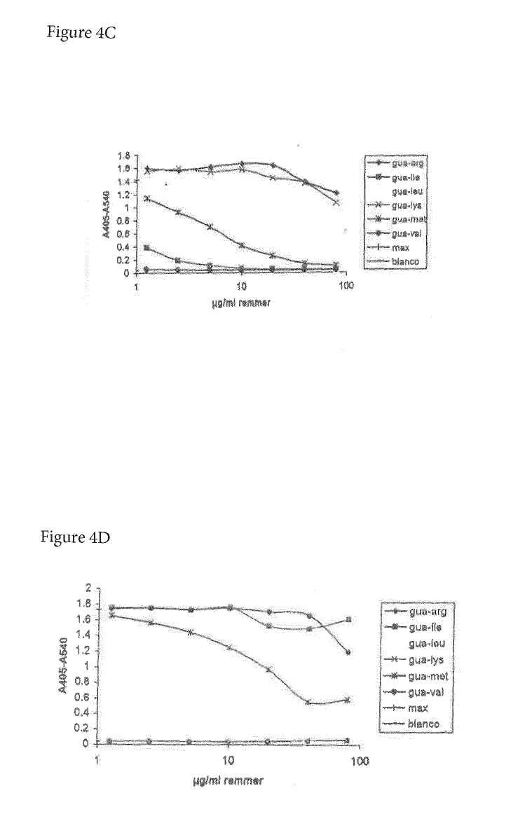

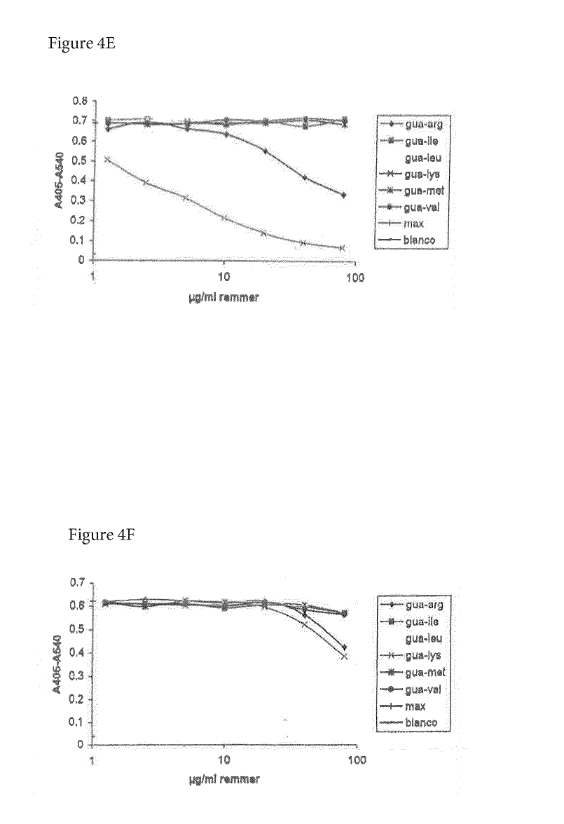

Guamerin and guamerin mutants expressing yeast strains were made in the same way as for fahsin. The guamerin Lys mutant was shown to strongly inhibit elastase (FIG. 2 and FIG. 4C). In FIG. 4 a summary is given of the inhibiting effects of guamerin and its mutants on 6 different proteinases.

For these assays 25 μl of mutant sample in PBS/0.2% Tween 20 or a dilution thereof in the same solvent was preincubated with 25 μl of proteinase for 60 minutes at 37° C. Then suitable substrate was added (50 μl) and the mixture was allowed to react for 1, hr (elastase, trypsine), 2 hrs (cathepsin G, chymotrypsin, thrombin) or 4 hrs (plasmin) at 37° C.

After incubation the absorbance was measured at 405 and 540 Angstrom and the difference of these values was plotted in FIGS. 4A-F.

1. Fahsin for use in the prevention of lung disease, selected from the group of emphysema, COPD and lung cancer.

2. Fahsin for use in therapy or prophylaxis of lung disease, particularly emphysema, through inhalation, specifically through inhalation of smoke, more particularly tobacco smoke.

3. Smoking article, such as a cigarette, including an e-cigarette, pipe tobacco, cigar or joint, for use in the therapy or prophylaxis of emphysema.

4. Smoking article according to claim 3, wherein said article comprises an elastase inhibitor, preferably selected from the group of fahsin, guamerin, piguamerin, hirustasin, bdellastasin and mutants of guamerin, piguamerin, hirustasin and bdellastasin that contain a leucine residue after the 6^{th} cysteine residue.

5. Smoking article according to claim 4, wherein the elastase inhibitor is included in tobacco or cannabis, either as a blend or as a protein expressed by said tobacco or cannabis.

6. Smoking article according to claim 4, wherein the cigarette is a filter cigarette or an e-cigarette and the elastase inhibitor is present in the filter.

7. Smoking article according to claim 4, wherein the smoking article is a cigarette or a joint and the elastase inhibitor is present in the cigarette paper.

8. Smoking article according to any of claims 4-6 wherein the elastase inhibitor is recombinantly produced fahsin.

9. Transgenic tobacco or cannabis comprising an elastase inhibitor, preferably fahsin.

10. Cigarette comprising the transgenic tobacco or cannabis according to claim 9.

11. Method to prevent or reduce emphysema, COPD or lung cancer, or to ameliorate a condition of the lungs, comprising smoking an smoking article according to any of claims 3-8.

12. Method to prevent or reduce emphysema, COPD or lung cancer, or to ameliorate a condition of the lungs, comprising inhaling an elastase inhibitor, preferably selected from the group of fahsin, guamerin, piguamerin, hirustasin, bdellastasin and mutants of guamerin, piguamerin, hirustasin and bdellastasin that contain a leucine residue after the 6^{th} cysteine residue, more preferably fahsin.

13. Method to improve the lung function of smokers by administration of an elastase inhibitor, preferably by administration of said inhibitor by inhalation.

14. Method according to claim 13, wherein said inhalation is inhalation of smoke wherein said smoke comprises said inhibitor.

15. Method according to claim 13 or 14, wherein said inhalation is inhalation through an e-cigarette, or wherein said smoke is generated by an e-cigarette.

16. Method according to any of claims 13 to 15, wherein said elastase inhibitor is selected from the group of fahsin, guamerin, piguamerin, hirustasin, bdellastasin and mutants of guamerin, piguamerin, hirustasin and bdellastasin that contain a leucine residue after the 6^{th} cysteine residue

17. A mutant of guamerin, that contains a leucine, arginine, isoleucine, lysine or valine residue after the 6^{th} cysteine residue.

18. A mutant of piguamerin, that contains a leucine, isoleucine, methionine, lysine or valine residue after the 6^{th} cysteine residue.

19. A mutant of hirustasin that contains a leucine, isoleucine, methionine, lysine or valine residue after the 6^{th} cysteine residue.

20. A mutant of bdellastasin that contains a leucine, arginine, isoleucine, methionine or valine residue after the 6^{th} cysteine residue.

21. A mutant of fahsin that contains an arginine, isoleucine, methionine, lysine or valine residue after the 6^{th} cysteine residue, preferably wherein said mutant contains an arginine residue.

22. A mutant of fahsin according to claim 21 for use in therapy, preferably for therapy or prophylaxis of pancreatitis, inflammation, edema, photo-aging or thrombosis.
